(19) United States
(12) Patent Application Publication
(10) Pub. No.: US 2007/0041950 A1
(43) Pub. Date: Feb. 22, 2007

(54) Method and device for selective addition of a bioactive agent to a multi-phase implant

(75) Inventors: Neil C. Leatherbury, San Antonio, TX (US); Joseph D. Blandford, San Antonio, TX (US)

Correspondence Address: GREENLEE WINNER AND SULLIVAN P C, 4875 PEARL EAST CIRCLE, SUITE 200, BOULDER, CO, 80301, US

(73) Assignee: OsteoBiologics, Inc., San Antonio, TX (US)

(21) Appl. No.: 11/345,115

(22) Filed: Feb. 1, 2006

Related U.S. Application Data
(60) Provisional application No. 60/649,418, filed on Feb. 1, 2005.

(51) Int. Cl. A61K 35/12; A61F 2/06

(52) U.S. Cl. 424/93.7; 424/426; 623/1.11

(57) ABSTRACT

A method and device for assembling a multi-phase implant for insertion into a patient defect site that spans more than one tissue type. The assembly device comprises a base and cover portion for constructing a two-phase implant separated by a membrane. A repeating intermediate portion that connects the base and cover can be used to construct implants with more than two phases, with each phase separated by a membrane. The invention is also for multi-phase implants, wherein adjacent phases are separated by a membrane. In an embodiment, the implant phases are selectively loaded with a bioactive agent selected to promote tissue repair in the tissue surrounding each phase.

### CROSS-REFERENCE TO RELATED APPLICATIONS

This application claims the benefit of U.S. Provisional Application No. 60/649,418, filed Feb. 1, 2005, hereby incorporated by reference to the extent not inconsistent herewith.

### BACKGROUND OF THE INVENTION

This invention relates to an apparatus and methods for performing repairs on defect sites that span more than one kind of tissue.

It is well known in the art that implants can be inserted into damaged bone or cartilage layers to treat injuries to those tissue layers. One type of implant procedure involves inserting plugs of healthy bone or cartilage that are harvested from a healthy area of the patient's body and transplanted into the defect, as disclosed in U.S. Pat. No. 5,152,763 (Johnson et al.), U.S. Pat. No. 5,919,196 (Bobic et al.), and U.S. Pat. No. 6,358,253 (Torrie et al.). In the alternative, an implant can consist of synthetic material, such as porous biocompatible foams or polymers, for example as disclosed in U.S. Pat. No. 4,186,448 (Brekke et al.), U.S. Pat. No. 5,607,474 (Athanasiou et al.), and U.S. Pat. No. 5,716,413 (Walter et al). patent application Ser. No. 10/785,388, filed Feb. 23, 2004, entitled “Bone and cartilage implant delivery device”, and U.S. Ser. No. 11/290,142 filed Nov. 30, 2005, entitled “Implants and delivery system for treating defects in articulating surfaces”, hereby incorporated by reference, disclose a delivery method and device for implanting material into a tissue defect.

Tissue defects can be repaired using tissue engineering scaffolds. For optimal repair and healing, such scaffolds generally require the addition of bioactive agents (e.g. cultured cells, growth factors, proteins, peptides, autologous agents, xenogenic agents, and/or allogenic agents). Particular tissue engineering difficulties can occur where a tissue defect spans different types of tissues or tissue regions. U.S. Pat. No. 5,981,825. In such cases, the biological conditions associated with each tissue region can be significantly different. Simultaneous repair of adjacent tissues can require delivery of different bioactive agents by different regions of the implant. This presents specific engineering problems for implants spanning multiple tissue regions. To maximize the implant's effectiveness at repairing the defect, the implant should have different phases, wherein each phase is tailored to the particular tissue that surrounds that phase. For instance, the mechanical properties (e.g. the stiffness) of the implant can be tailored to match those of the surrounding tissue. Also, the growth conditions may be different in each tissue region so that different types and/or amounts of growth factors should be specifically confined within each implant region. For some cases, different bioactive agents are required in the two regions. In addition, it may sometimes be necessary that a bioactive agent in the first implant region be excluded from the second implant region. The physical properties of each tissue region may be different, necessitating that the material used to construct each phase be different.

For example, implants for osteochondral repair must treat both a bone and cartilage region. For such repair regions, it may be desirable to selectively add a bone morphogenic protein (BMP), or other bioactive agents including, but not limited to, a cocktail of growth factors to the region of the implant to be inserted into the bone region and to add expanded cultured chondrocytes to the region of the implant to be inserted into the cartilage region. It can be advantageous to exclude the BMP from the chondrocytes to avoid chondrocyte transformation into hypertrophic chondrocytes or osteoblasts, thus forming bone in the cartilage region. In addition, for optimum cartilage formation the expanded cultured chondrocytes should be maintained at a high concentration within a limited volume. Thus, implants that span a bone and cartilage region should optimally be constructed as two phases, wherein there is no communication between the two phases for a defined period of time. The devices and methods of the present invention address the need in the art to construct multi-phase implants, tailored for the multi-tissue region defect site to be repaired.

### SUMMARY OF THE INVENTION

The present invention provides a multi-phase implant assembly device and methods for preparing multi-phase implants to correct tissue defect sites in patients that can span more than one type of tissue. The multi-phase implants can be used for a variety of applications, including osteochondral repair, bone-ligament reattachment, muscle-ligament attachment, muscle-bone attachment and other non-orthopedic applications. The different types of tissues to be repaired include, but are not limited to, bone, tendon, cartilage, muscle, ligament, nerve and skin.

The multi-phase implant device or “implant assembly” can be used to construct a two-phase implant, wherein each phase is separated by a membrane. The implant assembly is also used to construct a three-or-more-phase implant, wherein adjacent phases are separated by a membrane. In an embodiment, the implant assembly comprises a base and a cover. The base comprises a bottom portion, a top portion and a lower implant compartment having a cross-sectional shape that extends through the top and bottom portions. The cover is secured to the base and the cover comprises an upper portion having an upper implant compartment, wherein the upper implant compartment has a cross-section equivalent to the cross section of the base lower implant compartment. The cover further comprises a lower portion having a lower receiving compartment shaped to receive the top portion of the base thereby securing the cover to the base. “Equivalent” is used herein to specify that the implant phase contained in the upper implant compartment and the implant phase contained in the lower implant compartment substantially align with respect to each other such that the assembled implant can be removed from the implant assembly by applying a force to one end of the implant. If the cross-section is not equivalent, the phases are said to be unaligned such that applying a force to one end of the implant to remove the implant from the assembly results in significant deformation, tearing and/or damage to the implant. The cross-section of the receiving compartment encompasses the cross-section of the lower and upper implant compartments.

An embodiment of the implant assembly further comprises a well located at the top surface of the base lower implant compartment (e.g. the face that opposes the bottom surface of the cover when the cover is secured to the base). This well is designed to receive a membrane that separates each of the adjacent implant phases. In an embodiment, an o-ring is positioned on the surface of the top portion of the base for contacting a membrane positioned between the cover and the base. Another embodiment is an o-ring on a surface of the lower portion of the cover for contacting a membrane positioned between the cover and the base. In an embodiment both o-rings are used such that one o-ring is between the membrane bottom surface and base top surface and another o-ring is between the membrane upper surface and the cover bottom surface. In an embodiment, one or more of the o-rings are used in combination with the well for contacting a membrane positioned between the cover and the base. In an embodiment, no o-rings and no well is used, so that the membrane is positioned and held between the implant compartments by securing the cover to the base, thereby holding the membrane in place. The well depth can be less than the membrane thickness to facilitate sealingly connecting the base to the cover.

In an embodiment the cover is secured to the base by one or more welds, adhesives, and fastening means on the base removably engaged with fastening means on the cover. Fastening means that are removably engagable include, but are not limited to, snap beads and threaded connections.

In an embodiment, the fastening means comprises a snap bead, wherein the snap bead comprises a circumferential undercut located between the base bottom portion and base top portion; compartment overhang located at the bottom of the cover lower portion. The compartment overhang can snap into the base circumferential undercut, thereby engaging the cover lower portion to the circumferential undercut. A plurality of slots positioned along the cover lower portion facilitates sealingly connecting the cover lower portion to the circumferential undercut. These slots facilitate radial deformation, and radial recovery, of the cover lower portion as the lower portion passes over the base top portion and snaps into place upon the compartment overhang reaching the circumferential undercut.

In an embodiment, the implant compartment's cross-sectional shape can have any user-defined shape. To facilitate removal of a multi-phase implant contained within the assembly device by applying a force to one end of the multi-phase implant, in an embodiment an implant compartment's cross-sectional area and shape is constant over the implant height and equivalent to the cross-sectional area and shape of an adjacent implant compartment. The cross-section of the implant compartment can be, for example, square, hexagonal, triangular, rectangular, oblong, or any shape best suited to repair a tissue defect site. In an embodiment, the cross-section of the implant compartment is circular. In an embodiment, the implant cross-section can vary with implant height (e.g. instead of a cylinder, the implant can be “cone” shaped).

Any of the implant assemblies disclosed herein can contain a membrane having two surfaces, an upper surface facing the upper implant compartment and a lower surface facing the lower implant compartment. In the two-phase embodiment the membrane is located between the base opposing faces of the base and the cover. In an embodiment the membrane is positioned and secured using any one or more of a well and one or two o-rings. The membrane can be impermeable, selectively or partially permeable or permeable. In an embodiment the membrane is impermeable. In an embodiment the membrane is permeable or selectively permeable. In an embodiment the membrane is selectively permeable. In an embodiment the membrane is permeable.

Any of the implant assemblies disclosed herein further comprise a first phase of an implant located within the lower compartment and attached to the lower surface of the membrane, and/or a second phase of an implant located within the upper compartment and attached to the upper surface of the membrane. In an embodiment the first and second phases comprise materials having the same composition. In an embodiment the first and second phases each comprise different materials. The phases can be three-dimensional matrices such as scaffolds to facilitate cell growth or controlled release of growth factors or pharmaceutical drugs. In an embodiment the first phase is adapted for bone implantation and the second phase is adapted for cartilage implantation. As used herein, “adapted” refers to varying the characteristics of the phase to match the characteristics of the tissue in which the phase is to be implanted. In one embodiment, the characteristics that are matched are mechanical characteristics as disclosed in U.S. Pat. No. 5,607,474, and can be one or more of porosity, stiffness and compressibility properties.

In another embodiment, any of the implant assemblies having implant phases therein can contain one or more bioactive agent. “Bioactive agent,” as used herein, is used very broadly to refer to any substance that has a biological effect. The bioactive agent can include those that promote or inhibit cellular growth, migration, extracellular matrix deposition or cellular expression of RNA or protein. The bioactive agent can be naturally occurring or can be a pharmaceutical drug. The bioactive agent can include any cell type, including but not limited to, osteoblasts, chondrocytes, fibroblasts, muscle cells (smooth, cardiac and/or skeletal), nerve cells, epithelial cells, endothelial cells, and any combination or subcombination thereof. A bioactive agent also encompasses components of extracellular matrix, such as demineralized bone, processed tissue, elastin, collagen or cartilage matrix. In one embodiment the bioactive agent is a growth factor, pharmaceutical drug or suspension of cells. The bioactive agent may be autologous, allogenic or xenogenic agent. In an embodiment, the bioactive agent is selected from the group consisting of growth factors, extracellular matrix, pharmaceutical drugs, and suspensions of cells.

In another embodiment, the implant assembly device is for constructing an implant with three or more phases. This assembly is similar to the two-phase implant assembly in that it can include a base and cover, as discussed for the two-phase implant. An added component for a three-phase implant is one or more intermediate units positioned between the cover and the base. In an embodiment, the intermediate has a lower portion similar to the lower portion of the cover, and an upper portion similar to the top portion of the base. An intermediate implant compartment spans the upper portion of the intermediate part. An intermediate receiving compartment spans the lower portion of the intermediate part and in an embodiment, has a cross-sectional shape and area equivalent to the implant compartments contained in the base, cover and any other intermediate units. In an embodiment, the intermediate upper portion is secured to the cover. The intermediate upper portion comprising an intermediate implant compartment having a cross-sectional shape equivalent to the cross-sectional shape of the lower implant compartment, an upper intermediate portion sized to engage the lower receiving compartment of the cover, and a port running from outside the intermediate unit to the intermediate implant compartment for the addition or introduction of one or more bioactive agents to the intermediate implant compartment. The intermediate lower portion is secured to the base, the intermediate lower portion having an intermediate receiving compartment sized to receive the base top portion. In an embodiment, the implant assembly comprises two or more identical intermediate units.

The three-or-more phase implants, in an embodiment, further comprise a membrane positioned between each of the adjacent implant compartments. The membrane can be received by a well located at the top of the intermediate implant compartment. In an embodiment, any of the implant assemblies further comprise an o-ring on a top surface of the intermediate upper portion for contacting a membrane positioned between the intermediate portion and the cover and an o-ring on the bottom surface of the intermediate lower portion for contacting a membrane positioned between the intermediate portion and the base.

In an embodiment, the intermediate unit is secured to the base and the cover by one or more of welds, adhesives, and fastening means on the base removably engaged with fastening means on the cover, as discussed previously for the two-phase implant.

Any number of intermediate parts can be stacked between the base and cover to assemble an any-number-phase implant. An intermediate implant phase can be received by the intermediate implant compartment prior to assembling the intermediate part with the cover and the base. A needle port can be located through the outside wall of the intermediate part to communicate with the intermediate implant compartment. This port can facilitate the addition of one or more bioactive compounds suspended in a fluid to the intermediate phase. The port can be a hole or a septum for injecting fluid into the intermediate implant phase. A second port can be located through the outside wall of the intermediate part to provide an escape path for air contained within the intermediate implant void volume that is displaced by the inserted fluid. Alternatively, the intermediate implant can be loaded with a bioactive compound prior to assembly with the cover and the base, including by centrifugal introduction. For assistance in identifying the phase to which a bioactive compound should be added, each of the intermediate parts contained within the implant assembly device can be color-coded. Any one or more of the cover, base and intermediate parts can have a means for visually ensuring the implant is appropriately situated. Such means includes a transparent window, a slot, or at least a portion of the holder comprising a transparent material.

The multi-phase implant and associated holder can be placed into a suitable holder that mates the multi-phase implant to a delivery device. Such a holder is a convenient means for inserting the multi-phase implant into an appropriate delivery device. Alternatively, the implant can be removed from the holder and then loaded into the delivery device without using a holder. In an embodiment, the multi-phase implant is disposed within a delivery device. Such delivery devices are known in the art, and include U.S. patent application Ser. Nos. 10/785,388 and 11/290,142, hereby incorporated by reference. Briefly, the delivery device comprises a tubular outer shaft and an inner shaft. The tubular outer shaft having a proximal and distal end, a longitudinal axis, and an internal bore along the longitudinal axis of the outer shaft. The inner shaft having a distal end and a proximal end suitable for insertion into a defect, said inner shaft adapted to fit within said internal bore of the outer shaft so that the inner shaft and the outer shaft are slidably engaged. The implant is then ready for insertion, by the delivery device, into a patient tissue defect.

In an embodiment, the implant assembly for constructing a multi-phase implant comprises a base and a cover. The base comprises a bottom portion, a top portion and a lower implant compartment having a cross-sectional shape that extends the length of the base. The cover comprises an upper portion having an upper implant compartment, wherein the upper implant compartment cross-sectional shape is equivalent to the cross-sectional shape of the lower implant compartment, and a lower portion having a lower receiving compartment sized to receive the top portion of the base. The assembly also comprises means for positioning a membrane between the base and cover portions located on a top surface of the base and means for sealingly engaging the base with the cover.

The means for positioning the membrane includes a well, having a depth less than the thickness of the membrane, on one or more of the base and cover. Such a well allows the membrane to be positioned, and assists in keeping the membrane in place while the base and the cover are engaged. Another means for positioning utilizes one or more o-rings, wherein the o-rings are located between the membrane top surface and cover and/or the membrane bottom surface and the base. These o-rings facilitate appropriate positioning of the membrane by minimizing stress and associated deformation and compression when the base and cover are engaged. Another means for positioning utilizes both a well and one or more o-rings. Means for positioning also includes no well or no o-rings and refers to an appropriately-sized and composition membrane that is held between opposing parallel faces from the top of the base and bottom of the cover, wherein the membrane does not does not excessively deform when the base and cover engage.

The means for sealingly engaging the base with the cover includes a snap bead, threaded connection, thermoplastic sealed by ultrasonic welding and adhesives. Any of these means are suitable for sealingly engaging the base and cover such that the presence of the membrane along with the engagement of the base and cover prevents fluid from transiting from one compartment to another compartment or leaking out of the assembly between the cover and the base.

In an embodiment, the implant assembly further comprises one or more intermediate units placed between the base and cover. The intermediate unit comprises an intermediate upper portion and an intermediate lower portion. The intermediate upper portion comprises an intermediate implant compartment having a cross-sectional shape equivalent to the cross-sectional shape of the lower implant compartment, an upper intermediate portion sized to fit into the lower receiving compartment of the cover, means for positioning a membrane between said intermediate implant compartment and an adjacent compartment located on the top surface of the upper intermediate portion, and means for the intermediate upper portion to sealingly engage the cover lower portion. The intermediate lower portion comprises an intermediate receiving compartment sized to receive the base top portion, and means for the intermediate lower portion to sealingly engage with the base. Optionally, the intermediate unit further comprises a needle port through the intermediate upper portion for adding and introducing a bioactive agent to the intermediate implant compartment.

Means for positioning a membrane between said intermediate implant compartment and an adjacent compartment are as described for the two-phase (base and cover) implant, and includes a well, an o-ring, combination of a well and an o-ring, and a pair of opposing parallel faces, that when engaged, position the membrane.

Means for the intermediate upper portion to sealingly engage the cover lower portion and means for the intermediate lower portion to sealingly engage with the base are as described for the two-phase (base and cover) implant, and includes a snap bead, threaded connection, thermoplastic sealed by ultrasonic welding and adhesives.

An embodiment of the present invention is a multi-phase implant made by any of the implant assemblies disclosed herein, wherein adjacent phases are separated by a membrane. The implant is a two-phase implant having one membrane. The implant is a three-phase implant having two membranes. The implant is a more than three phase implant having more than two membranes. Each phase can contain one or more bioactive agents. In an embodiment, each phase is adapted for insertion into a particular tissue. In an embodiment, the membrane separating the adjacent phases has a thickness between 125 μm and 250 μm.

A further embodiment is an implant adapted for insertion into a tissue defect, said implant comprising a membrane having a top surface and a bottom surface, with a first implant phase attached to the membrane top surface and a second implant phase attached to said membrane bottom surface.

In an embodiment, the first implant phase has a composition that is different than the second implant phase composition. In a further embodiment, the first implant phase and/or second implant phase of the implant contains one or more bioactive agent.

Each of the implant phases are adapted for implantation into a specific tissue. In one embodiment the first implant phase is adapted for bone implantation and the second implant phase is adapted for cartilage implantation. In one embodiment, the adaptation is matching mechanical properties of the implant phase to the mechanical properties of the tissue in which each phase is implanted. The mechanical properties include, but are not limited to, porosity, compressibility and stiffness. In addition, the bioactive agents are selectively loaded into a phase so as to maximize repair of the particular tissue surrounding that phase.

The invention is also a method for constructing two-phase implants and three-or-more-phase implants, wherein each phase is separated from an adjacent phase by a membrane. In an embodiment, the method is for constructing a two-phase implant with the two phases separated by a membrane, comprising providing a first holder for a first implant phase, also comprising a holder for a membrane at a first end of the first holder, providing a second holder for a second implant phase, inserting a first implant phase into said first holder, inserting a membrane into said first end of the first holder and attaching the membrane to said first phase; and inserting a second implant phase into said second holder and attaching it to said membrane to construct a two-phase implant with the two phases separated by a membrane.

In an embodiment, the method is constructing a two-phase implant, with a first (e.g. a base) and a second (e.g. a cover) holder containing a first and a second implant phase. The first implant phase optionally contains a well at one end for receiving a membrane. A membrane is inserted into the well of the first holder and attached to the first implant phase. The second implant phase, contained in a second holder is attached to the membrane. The first and second holders are sealingly engaged. In this manner, the implant comprises a first and second phase, with the two phases separated by a membrane.

A three-or-more-phase implant is similarly constructed by using one or more intermediate parts, in addition to a cover and a base. An intermediate (e.g. third) phase is inserted into the one or more intermediate implant compartments and attached to the lower face of the intermediate membrane. The intermediate part with the intermediate (third) implant phase and membrane is then sealingly engaged to the base so that the intermediate implant phase attaches to the top surface of the membrane disposed in the base's well. The cover is then sealingly engaged to the top portion of the intermediate part. A first implant phase is inserted into the lower compartment and attached to the bottom surface of the membrane separating the intermediate (third) phase and the first phase. A second implant phase is inserted into the upper compartment and attached to the upper surface of the membrane separating the second phase and the intermediate (third) phase. By inserting additional intermediate parts, containing additional intermediate (e.g. 4^{th}, 5^{th}, etc.) phases between the cover and base, any-number-phase implants are constructed.

In an embodiment, the method further comprises sealingly engaging the first and second holders. In an embodiment, the method further comprises inserting a membrane that is impermeable or selectively permeable.

In an embodiment, the method further comprises inserting one or more bioactive agents into the first implant phase and inserting one or more bioactive agents into the second implant phase. In an embodiment the bioactive agent is applied to the phase before the phase is inserted into the device. In an embodiment the bioactive agent is applied to the phase after the phase is inserted into the device. In an embodiment, the one or more bioactive agents in the first phase promote a first tissue repair by selectively delivering the bioactive agent in the first implant phase to the first tissue and the one or more bioactive agents in the second implant phase promote a second tissue repair by selectively delivering the bioactive agent in the second implant phase to the second tissue. In an embodiment, the first tissue is cartilage and the second tissue is bone. In an embodiment, the one or more bioactive agents in the phase to promote cartilage repair comprises a suspension of chondrocytes.

The method further comprises inserting an implant into a delivery device. In an embodiment, the method further comprises inserting the implant from the delivery device into a defect in a patient. In an embodiment, the defect in a patient spans bone tissue and cartilage tissue.

### BRIEF DESCRIPTION OF THE DRAWINGS

FIG. 1 shows a base embodiment from: (A) top view; (B) perspective view; (C) cross-sectional view along line labeled C-C in FIG. 1A;, and (D) side view.

FIG. 2 shows a cover portion embodiment from: (A) top view; (B) perspective view; and (C) cross-sectional view along the line labeled C-C in FIG. 2A.

FIG. 3 shows an implant assembly for constructing a two-phase implant from: (A) top view; (B) perspective view; (C) cross-sectional view along the line labeled C-C in FIG. 3A; and (D) cross-sectional view of a second embodiment along the line labeled C-C in FIG. 3A. The open arrows in (C) and (D) indicate the cover and base sealingly engage one another. For clarity, the base and cover are not sealingly engaged in (B), (C) and (D).

FIG. 4 shows a three-phase implant assembly device. The open arrows indicate each of the cover and base sealingly engages to the intermediate unit.

FIG. 5 shows an implant contained within the assembled device of FIG. 3 being loaded into a delivery device by a holder. (A) Shows the implant assembly within a holder, ready to be loaded into a delivery device. (B) Shows the implant after it has been loaded into the delivery device.

### DETAILED DESCRIPTION OF THE INVENTION

The invention may be further understood by the following non-limiting examples. Although the description herein contains many specificities, these should not be construed as limiting the scope of the invention but as merely providing illustrations of some of the presently preferred embodiments of the invention. For example, thus the scope of the invention should be determined by the appended claims and their equivalents, rather than by the examples given.

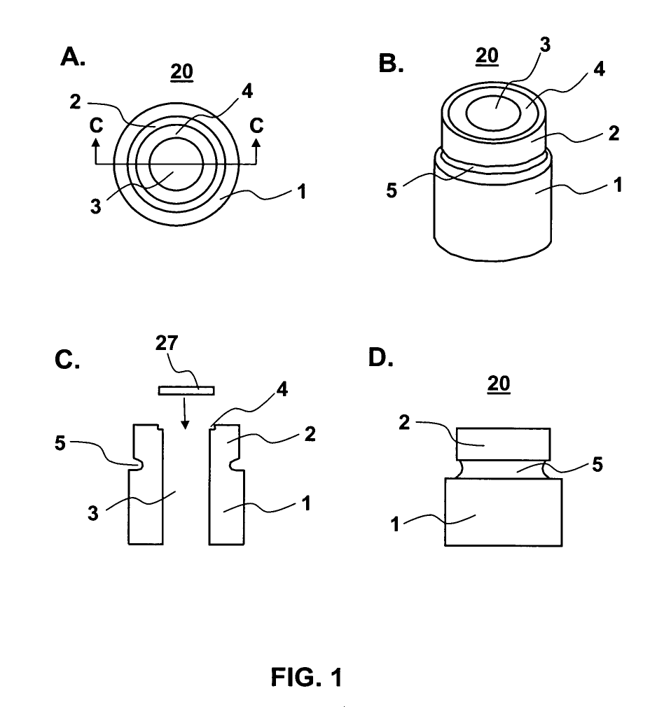

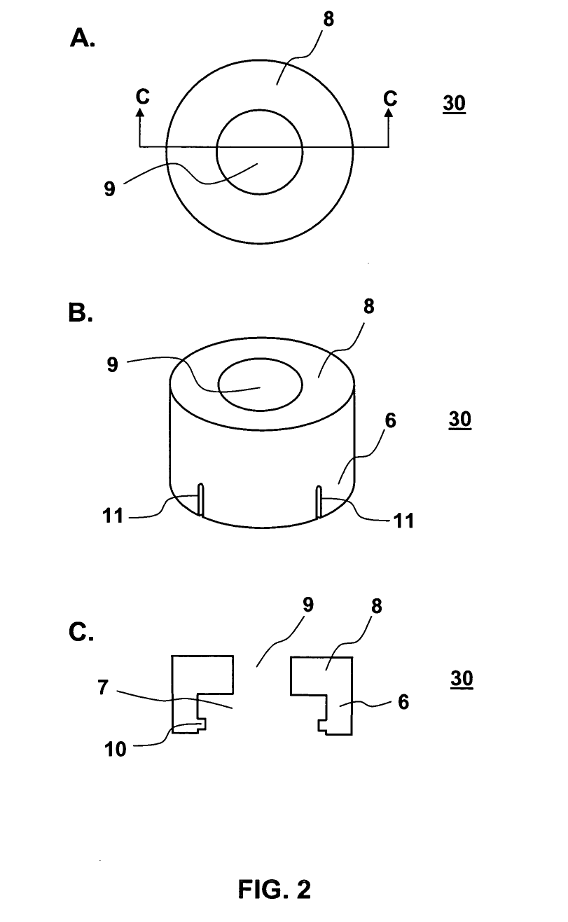

FIGS. 1A-1D show one embodiment of the base portion 20 of the multi-phase implant assembly. In an embodiment, the base 20 has a bottom portion 1, a top portion 2, and a cylindrically-shaped lower implant compartment 3 running the vertical length of the base. FIG. 1A, is a top view of the base, where the lower implant compartment 3 has a circular cross-section, centered within the base 20. In one embodiment, the top portion spans between about one-quarter and about one-third the vertical length of the base. The cross-section of the lower implant compartment 3 need not be circular, but can have any cross-section shaped to receive an implant suited for repair of the tissue defect. The external radius of the top portion 2 can be less than the external radius of the bottom portion 1. The top surface of the top portion 2 can have a shallow well 4 for receiving a membrane or a thin film 27, whose thickness can be greater than the well's depth. Optionally, the shape of the well 4 need not be circular, but can correspond to the lower implant compartment 3 cross-section. For example, if the cross-section of the implant compartment 3 is oblong, the well can have an oblong shape. Optionally, the base can have a circumferential undercut 5 for receiving a compartment overhang 10 located on the cover portion 30 (see FIG. 2), thereby engaging the base 20 to the cover 30.

FIGS. 2A-2C show one embodiment of the cover portion 30 of the multi-phase implant assembly. The cover portion can have a lower portion 6 and an upper portion 8. In an embodiment the lower portion spans about one-half the vertical length of the cover. In an embodiment the lower portion spans less than one-half the vertical length of the cover. The lower portion 6 defines a volume of a lower receiving compartment 7. The upper portion defines a volume of an upper implant compartment 9 that has the same cross-sectional shape as the cross-sectional shape of the base lower implant compartment 3. In a cylindrically-shaped lower compartment 3 cross-section embodiment, the inner radius of the upper portion 8 is of the same radius as the base lower implant compartment 3. Accordingly, the inner radius of the upper portion 8 defines an upper implant compartment 9 of the same radius as the lower implant compartment 3. In an alternative embodiment, the radius of the upper implant compartment 9 and/or the radius of the lower implant compartment 3 vary with the vertical height of the cover 30 and base 20, respectively.

A “snap bead” is one means for sealingly connecting the base and cover. As used herein, snap bead refers to the radius of the receiving compartment 7 being sufficiently expanded during the assembly process so that the base top portion 2 passes cover compartment overhang 10. Once the base top portion 2 passes cover compartment overhang 10, the radius of receiving compartment 7 returns back to normal as compartment overhang 10 snaps into circumferential undercut 5, thereby fitting the base's top portion 2 into the cover's receiving compartment 7. Optional slots 11 in the cover facilitate in sealingly connecting the base and cover by allowing a reversible radius increase as compartment overhang 10 passes over base top portion 2, and a corresponding radius decrease as compartment overhang 10 snaps into circumferential undercut 5. A snap bead mechanism is one means whereby the cover and base can sealingly engage each other.

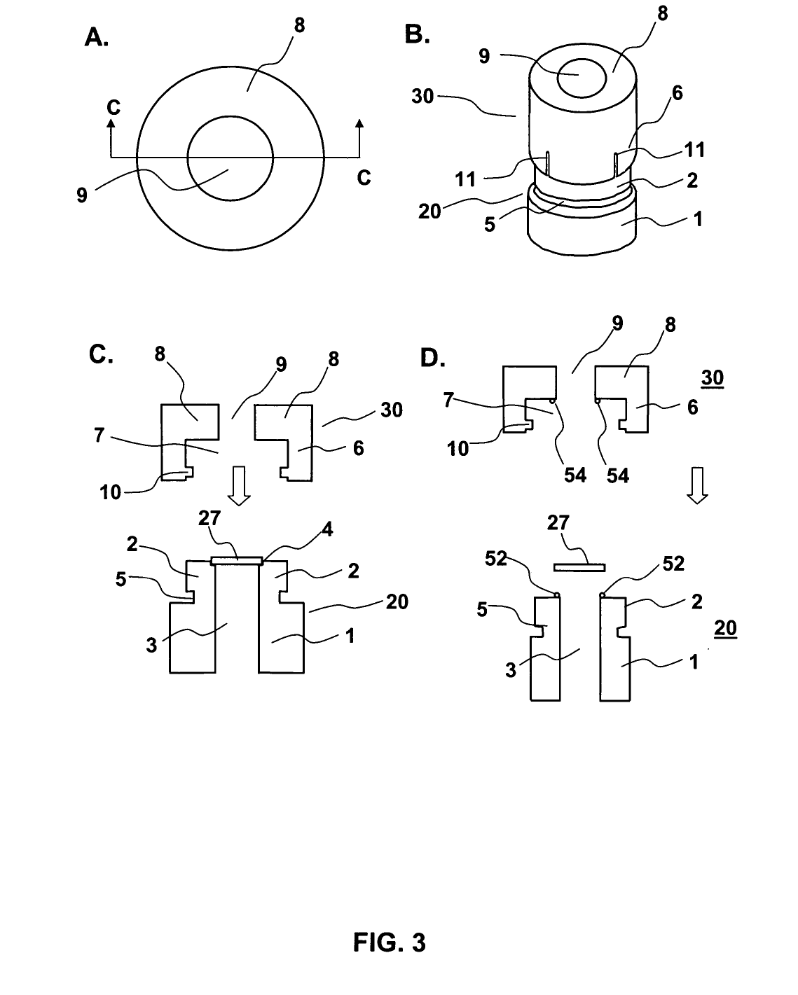

FIGS. 3A-3D show one embodiment of the base 20 (as in FIG. 1) engaging the cover 30 (as in FIG. 2). FIGS. 3C and 3D each show an embodiment for placing and securing a membrane 27 between the base 20 and cover 30. As shown in FIG. 3C, engagement of the base 20 and cover 30 form a cylindrical channel made up of a lower implant compartment 3 and an upper implant compartment 9. A membrane or thin film 27 can sit in the well 4, and when the base 20 and cover 30 engage, the cover lower portion 6 presses against the base top portion 2 and the membrane, thereby sealingly engaging the base and cover. In addition, the opposing faces comprising the bottom surface of 8 and top surface of 2, compress the edges of the membrane to form a seal between upper implant compartment 9 and lower implant compartment 3. FIG. 3D shows an embodiment with a first o-ring 52 on the top surface of the base top portion 2 and a second o-ring 54 on the bottom surface of the cover lower portion 6. O-rings 52 and 54 can be used to assist in sealingly engaging the membrane to the base 20 and cover 30 when the base and cover are secured to each other, thereby preventing fluid communication between the lower implant compartment 3 and the upper implant compartment 9. In an embodiment, one or both o-rings can be used in combination with the well to secure the membrane between the base 20 and cover 30. To assist in o-ring placement, o-rings can be placed in grooves made on the bottom surface of base upper portion 8 and top surface of cover top portion 2. O-rings can be used in addition to or instead of the well disposed within the base of the device. In an embodiment, the o-rings minimally protrude above the faces of the device and are made of a low-durometer rubber (i.e. less than 50 on a shore-D scale) to minimize the physical stress placed on the membrane during engagement of the base and cover. It is important that the membrane create a seal between the implant compartments without the membrane excessively deforming. This is accomplished by matching the thickness of the membrane to the gap associated with the mating components (e.g. the base and cover). Alternatively, the membrane can be secured between the base 20 and cover 30 without o-rings or a well by carefully compressing the membrane 27 between the parallel opposing faces of the cover 30 and base 20 without excessively deforming the membrane.

The particular means utilized to sealingly engage the base and cover is not important, so long as fluid cannot travel between the lower and upper implant compartment by bypassing the membrane. “Sealingly engaged” or “engaging,” as used herein, refers to the cover and base mating at the face formed by the top portion 2 and lower portion 6, thereby compressing the membrane, and creating a seal whereby fluid cannot leak around the membrane between the upper implant and lower implant compartments. The membrane can be sealingly engaged by compressing the membrane thickness between two opposing parallel faces, or by disposing an o-ring within one or both mating faces of the device to compress the membrane. The membrane can optionally be located within a well 4.

The membrane can be prepared by pressing a polymer of 85/15 DL-PLG (IV=0.76), or other suitable polymeric material, between two sheets of release paper forming a desired permeable, selectively permeable or impermeable barrier. Alternatively, the membrane can be prepared by extrusion, solvent casting, or injection molding the material. The permeability (as well as the permeability selectivity) of the membrane can be controlled by means known in the art (e.g. by affecting membrane porosity and/or pore size, charge, etc.). The membrane can be constructed to selectively permit the passage of certain substances through the membrane, while excluding passage of other substances that could detrimentally affect the other implant phase. The final thickness is preferably between 125 and 250 μm. A sharp punch can be used to cut a disk from the membrane sheet to fit the well 4 contained within the base. The membrane 27 can then be placed within the well 4, and the base and cover mated, thereby forming two compartments separated by the membrane. The membrane thickness and compressibility govern the well depth. For example, the well depth is less than the membrane thickness.

An implant phase is comprised of a material, preferably a polymeric material, material composite or transplanted biological tissue, and can be fitted to the upper compartment and to the lower compartment. An example of material suitable for an implant phase of the present invention can be a composite of 85/15 DL-PLG, calcium sulfate, PGA fibers, and a surfactant. Other materials known to the art may also be used. This material can be punched or otherwise shaped to match the cross-sectional shape of the implant compartment. The punched material can be shaped like a plug and can be of any length. In one embodiment, the material is between 1 mm and 18 mm. Other examples of materials suitable for making phases in an implant are known in the art, including implant materials discussed in the Background section. The implant material can be bioerodible.

The implant materials are generally prepared and cut to the appropriate size outside the assembly device. A solvent solution can be used to wet the to-be-attached surface of the prepared implant phase, thereby partially dissolving the surface of the polymer to facilitate adhesion of the membrane. The wetted prepared implant phase can then be inserted into the appropriate implant compartment. This process can be repeated for the opposing membrane surface and other implant compartment. The materials within each phase can have different or similar properties. For example, each phase can have different mechanical properties (e.g. elasticity and/or porosity) that match the mechanical properties of the tissue in which each phase is to be implanted. The phases can also be prepared from different materials.

A first implant phase can be attached to the membrane by any means known in the art, including solvent adhesion, thermal adhesion, ultrasonic welding, chemical reaction, or the like. To maintain the structural integrity of the membrane, the process of attachment should not perforate the membrane. Similarly, a second implant phase can be attached to the other surface of the membrane, thereby creating a two-phase implant separated by a membrane that can be impermeable, permeable, or selectively-permeable. After assembly of the two-phase and membrane implant, the assembled implant can be cured at 72° C. under vacuum for 24 hours to remove residual solvent, if necessary. The assembly device containing the multi-phase implant can be packaged and sterilized by means known in the art (i.e. ethylene oxide, gamma irradiation, e-beam).

The implant phases, within the assembled device, can be loaded with the desired bioactive agent(s). In an embodiment, the bioactive agent(s) are loaded under sterile conditions, after the assembled device and implant have been sterilized. In one embodiment the bioactive agent(s) can be suspended in a fluid so that the suspension can be applied to, and absorbed and/or attached by, the implant phase. Depending on the particular bioactive agent, the phases can be loaded using a syringe-type delivery device. In another embodiment, bioactive agents are introduced to an implant phase by centrifugation. The agent is dispensed to the intended implant phase contained in the implant assembly and the implant assembly placed in a centrifuge tube. The tube containing the implant assembly is spun in a centrifuge at an appropriate speed to ensure infiltration of the agent into the void spaces of the implant phase. The process is readily accomplished under sterile conditions to ensure continued sterility using techniques known to the art.

As an example of loading bioactive components, a suspension of cultured chondrocytes can be prepared in a carrier gel and placed over the cartilage phase of a two-phase implant. The gel itself may be biologically active or inert, for example the chondrocytes may be suspended in an autogenous fibrin gel. The cell suspension is dispensed into the upper well of the implant and either allowed to soak into the pores of the implant or is gently centrifuged to encourage migration into the pores. Depending on the vehicle used for suspending the cells, additional treatment may be applied to activate gelation. For example, thrombin may be added to a fibrinogen solution to create a fibrin gel, or calcium ion may be added to activate an alginate gel. Other gel activation techniques as known in the art may also be applied.

Other cell types and suspension solutions may used. For example mesenchymal stem cells, adipose derived stem cells, muscle derived stem cells, stem cells from banked cord blood, or embryonic stem cells can be used, either in a differentiated or undifferentiated state. Gel carriers can be prepared from gelatin, hyaluronan, cellulose derivatives, polyethylene oxide (PEO), polysaccharides, polypeptides, and derivatives or combinations of these components. The gel may be cross linked, thixotropic, or temperature, pH, or ion responsive.

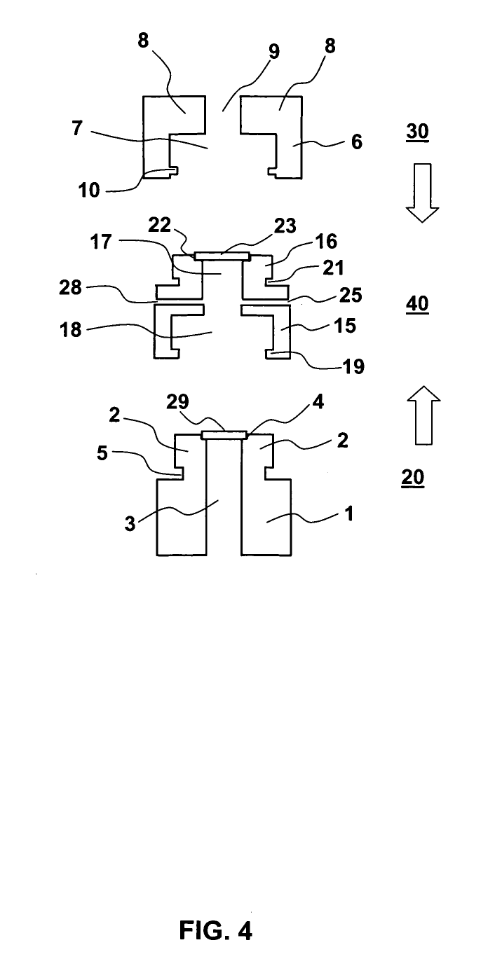

FIG. 4 shows another embodiment, wherein a three-phase implant can be constructed. As for the two-phase implant, the device includes a base 20 and a cover 30. An additional component is the intermediate unit 40. The intermediate unit includes an intermediate lower portion 15 and an intermediate upper portion 16. An intermediate implant compartment 17, with an identical cross-section as the lower 3 and upper 9 implant compartments, can be located within the intermediate upper portion 16. An intermediate receiving compartment 18 is located within the intermediate lower portion 15. The intermediate receiving compartment 18 is shaped to receive the top portion 2 of the base 20. Alternatively, for implants having more than three phases, the intermediate receiving compartment 18 can receive an intermediate upper portion 16 from a second intermediate part. Similarly, the intermediate upper portion 16 can be shaped to sealingly engage, in addition to base top portion 2, an intermediate lower portion 15.

As for the two-phase implant assemblies, the means to sealingly engage the intermediate portion to one of another intermediate portion, cover, or base can be a snap bead, threaded connection, thermoplastic sealed by ultrasonic welding or adhesives. In one embodiment, snap beads are used as the sealingly engaging means. An intermediate compartment overhang 19 can be located at the bottom of the intermediate part 40 to snap into circumferential undercut 5 (or alternatively, an intermediate circumferential undercut 21 of another intermediate part), thereby sealingly engaging the intermediate part with the base (or another intermediate unit). An intermediate circumferential undercut 21 can be positioned along the intermediate upper portion 16 for sealingly engaging the cover 30 (or another intermediate lower portion 15). The intermediate part can have an intermediate well 22 for receiving an upper-intermediate separating membrane 23. As with the two-phase implant shown in FIG. 3D, optional o-rings with or without well 22 can be used to sealingly engage the intermediate unit. The intermediate part 40 can also contain a needle port 25 for the addition of one or more bioactive agents, suspended in fluid, to the intermediate implant phase. An additional relief port 28 can be provided for displaced air from the intermediate implant volume to escape as fluid is injected through port 25, or for evacuation of the chamber containing the intermediate phase by application of negative pressure to draw the fluid into the implant material. Alternatively, the intermediate unit phase can be pre-loaded with a bioactive agent before placement between the cover and base, thereby obviating the need for ports 25 and 28. The base can have a well 4 for receiving an intermediate-lower separating membrane 29. In this manner an implant with the following regions can be constructed: first phase-membrane-intermediate (third) phase-membrane-second phase. The membranes need not be of identical composition or physical properties (e.g. one can be permeable, and the other can be impermeable). For the snap-bead embodiment, the minimum height of an intermediate part is constrained by the size of the snap-bead, so that the intermediate part can be at least approximately 1 mm in height.

In this embodiment, any number of intermediate parts 40 can be connected to each other, with a base at one end and a cover on the opposite end, thereby forming an implant with any number of phases. Bioactive agent(s) can be selectively loaded onto the desired implant phase through a needle port 25, thereby tailoring the biological conditions of each implant phase to the tissue conditions that will surround each implant phase.

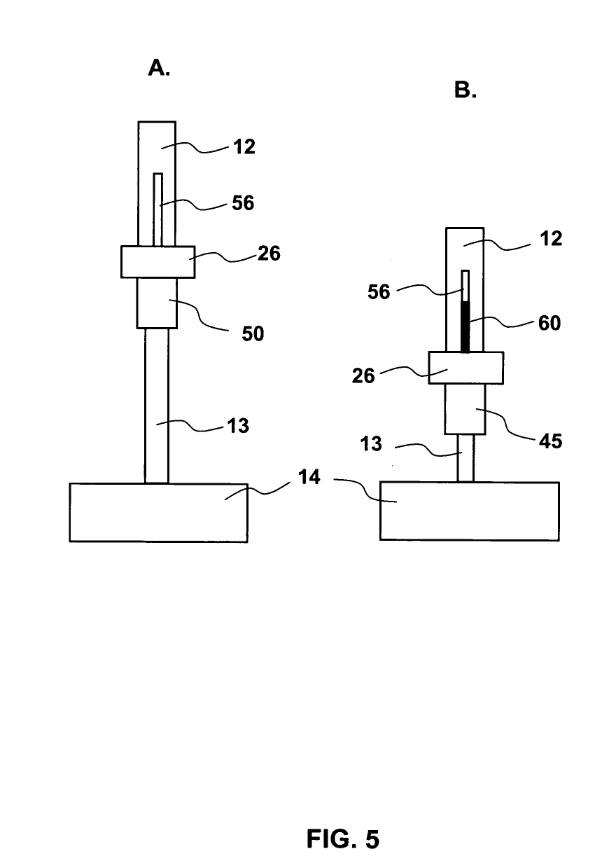

FIGS. 5A-5B illustrate a one-step process for loading the implant contained within the assembly device into a delivery device. The implant-loaded assembly devices can be disposed in a loading device comprising a post 13 whose dimensions match the dimension of the upper and lower implant compartments, and a holder 26. The holder 26 temporarily connects one end of the implant-loaded assembly within a holder 50 to a delivery device 12. Pressing on the delivery device 12 forces the implant-loaded assembly 50 down the post 13, thereby breaking and freeing the membrane from the implant assembly and forcing the multi-phase implant 60 into the delivery device 12. In a preferred embodiment the post 13 is supported by a platform 14. Alternative holders suitable for loading an implant into a delivery device are disclosed in U.S. patent application Ser. No. 10/785,388. The delivery device can have a means for visualizing the implant, e.g. a viewing window 56, or a transparent surface to assist the user in verifying the implant is properly loaded and ready for delivery. The implantless assembly device, contained within holder 45, can be discarded or disassembled, cleaned and reused. The loaded delivery device 12 is then ready to deliver the implant to repair a tissue defect. Delivery devices are known in the art and can include those disclosed in patent application Ser. No. 10/785,388, filed Feb. 23, 2004, and U.S. Ser. No. 11/290,142 filed Nov. 30, 2005, U.S. Pat. Nos. 5,782,835 and 6,395,011, hereby incorporated by reference, specifically for delivery devices and methods disclosed therein. Multiple phases of an implant can be fabricated, sterilized, and selectively loaded with bioactive agents within one implant assembly device of the present invention, and potentially delivered to a defect site, with each phase (and associated bioactive agents) selectively tailored to the tissue surrounding each phase.

The base and cover components can be prepared from metal, alloys, plastics, composites, or the like. For economic and convenience reasons, the components are preferably plastic. The components can be either machined or molded to create the form.

When a Markush group or other grouping is used herein, all individual members of the group and all combinations and subcombinations possible of the group are intended to be individually included in the disclosure.

Every formulation or combination of components described or exemplified can be used to practice the invention, unless otherwise stated. Specific names of compounds are intended to be exemplary, as it is known that one of ordinary skill in the art can name the same compounds differently. One of ordinary skill in the art will appreciate that methods, device elements, starting materials, synthetic methods and structures, other than those specifically exemplified can be employed in the practice of the invention without resort to undue experimentation. All art-known functional equivalents, of any such methods, device elements, starting materials, synthetic methods, and structure are intended to be included in this invention. Whenever a range is given in the specification, for example, a temperature range, a time range, a size range, or a composition range, all intermediate ranges and subranges, as well as all individual values included in the ranges given are intended to be included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations which is not specifically disclosed herein.

All references throughout this application, for example patent documents including issued or granted patents or equivalents; patent application publications; and non-patent literature documents or other source material, are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

We claim:

1. An implant assembly for constructing a two-phase implant comprising:
a. a base comprising:
i. a bottom portion;
ii. a top portion;
iii. a lower implant compartment having a cross-sectional shape, said lower implant compartment extending through said top portion and said bottom portion;
b. a cover secured to said base comprising:
i. an upper portion having an upper implant compartment, wherein the upper implant compartment cross-sectional shape is equivalent to the cross-sectional shape of the lower implant compartment; and
ii. a lower portion having a lower receiving compartment sized to receive the top portion of the base.

2. The implant assembly of claim 1 further comprising a well formed at the top of the lower implant compartment for receiving a membrane.

3. The implant assembly of claim 2 further comprising an o-ring on a surface of the top portion of the base for contacting a membrane positioned between the cover and the base.

4. The implant assembly of claim 3 further comprising an o-ring on a surface of the lower portion of the cover for contacting a membrane positioned between the cover and the base.

5. The implant assembly of claim 1, wherein the cover is secured to the base by one or more of welds, adhesives, and fastening means on the base removably engaged with fastening means on the cover.

6. The implant assembly of claim 5 wherein the fastening means comprises a snap bead, wherein said snap bead comprises:
a. a circumferential undercut, wherein said undercut is located between the base bottom portion and base top portion;
b. a compartment overhang, wherein said compartment overhang is located at the bottom of the cover lower portion, for engaging the cover lower portion to the circumferential undercut;
c. a plurality of slots positioned along the cover lower portion to facilitate engaging the cover lower portion to the circumferential undercut.

7. The implant assembly of claim 1 wherein said lower and upper implant compartment cross-sectional shape is circular.

8. The implant assembly of claim 1 further comprising a membrane located between the base and the cover, said membrane having a lower surface and an upper surface.

9. The implant assembly of claim 8 wherein said membrane is impermeable.

10. The implant assembly of claim 8 wherein said membrane is permeable or selectively permeable.

11. The implant assembly of claim 8 further comprising:
a. a first phase of an implant located within the lower compartment and attached to the lower surface of the membrane, and
b. a second phase of an implant located within the upper compartment and attached to the upper surface of the membrane.

12. The implant assembly of claim 11 wherein said first phase and said second phase each comprise different materials.

13. The implant assembly of claim 11 wherein said first phase is adapted for bone implantation and said second phase is adapted for cartilage implantation.

14. The implant assembly of claim 11 wherein the first phase and/or second phase contain one or more bioactive agent.

15. The implant assembly of claim 14 wherein the one or more bioactive agent is selected from the group consisting of growth factors, extracellular matrix, pharmaceutical drugs, and suspension of cells.

16. An implant assembly for constructing a three-or-more-phase implant comprising the device of claim 1, and further comprising one or more intermediate units positioned between the cover and the base, wherein the intermediate unit comprises:
a. an intermediate upper portion secured to the cover, the intermediate upper portion comprising:
i. an intermediate implant compartment having a cross-sectional shape equivalent to the cross-sectional shape of the lower implant compartment;
ii. an upper intermediate portion sized to engage the lower receiving compartment of the cover;
iii. a port for the addition of one or more bioactive agents to the intermediate implant compartment;
b. an intermediate lower portion secured to the base, the intermediate lower portion having an intermediate receiving compartment sized to receive the base top portion.

17. The implant assembly of claim 16 further comprising one or more additional intermediate units identical to said intermediate unit, positioned between said cover and said base.

18. The implant assembly of claim 16 further comprising a membrane between each of the adjacent implant compartments.

19. The implant assembly of claim 18 further comprising a well formed at the top of the upper intermediate compartment for receiving a membrane.

20. The implant assembly of claim 19 further comprising an o-ring on a top surface of the intermediate upper portion for contacting a membrane positioned between the intermediate portion and the cover and an o-ring on the bottom surface of the intermediate lower portion for contacting a membrane positioned between the intermediate portion and the base.

21. The implant assembly of claim 11, wherein said implant assembly is disposed within a loading device.

22. An implant assembly for constructing a multi-phase implant comprising:
a. a base comprising
i. a bottom portion;
ii. a top portion;
iii. a lower implant compartment having a cross-sectional shape, said lower implant compartment extending through said top portion and said bottom portion;
b. a cover comprising:
i. an upper portion having an upper implant compartment, wherein the upper implant compartment cross-sectional shape is equivalent to the cross-sectional shape of the lower implant compartment; and
ii. a lower portion having a lower receiving compartment sized to receive the top portion of the base;
c. means for positioning a membrane between the base and cover portions; and
d. means for sealingly engaging the base with the cover.

23. The implant assembly of claim 22 further comprising one or more intermediate units placed between the base and cover, wherein the intermediate unit comprises:
a. an intermediate upper portion comprising:
i. an intermediate implant compartment having a cross-sectional shape equivalent to the cross-sectional shape of the lower implant compartment;
ii. an upper intermediate portion sized to engage the lower receiving compartment of the cover;
iii. means for positioning a membrane between said intermediate implant compartment and an adjacent compartment;
iv. means for the intermediate upper portion to sealingly engage the cover lower portion;
b. an intermediate lower portion comprising:
i. an intermediate receiving compartment sized to engage the base top portion;
ii. means for the intermediate lower portion to sealingly engage the base.

24. A multi-phase implant made using the implant assembly of claim 1, wherein adjacent phases are separated by a membrane.

25. The implant of claim 24 wherein the adjacent phases are separated by a membrane having a thickness between 125 μm and 250 μm.

26. An implant for insertion into a tissue defect, said implant comprising:
a. a membrane having a top surface and a bottom surface;
b. a first implant phase attached to said membrane top surface; and
c. a second implant phase attached to said membrane bottom surface.

27. The implant of claim 26 wherein the first implant phase has a composition that is different than the second implant phase composition.

28. The implant of claim 26 wherein the first implant phase and/or second implant phase contain one or more bioactive agent.

29. The implant of claim 28 wherein the first implant phase is adapted for bone implantation and the second implant phase is adapted for cartilage implantation.

30. A method of constructing a two-phase implant with the two phases separated by a membrane, said method comprising:
a. providing a first holder for a first implant phase, also comprising a holder for a membrane at a first end of the first holder;
b. providing a second holder for a second implant phase;
c. inserting a first implant phase into said first holder;
d. inserting a membrane into said first end of the first holder and attaching the membrane to said first phase; and
e. inserting a second implant phase into said second holder and attaching it to said membrane
to construct a two-phase implant with the two phases separated by a membrane.

31. The method of claim 30 further comprising sealingly engaging said first and second holders.

32. The method of claim 31 wherein the membrane is impermeable or selectively permeable.

33. The method of claim 32 further comprising inserting one or more bioactive agents into the first implant phase and inserting one or more bioactive agents into the second implant phase.

34. The method of claim 33 wherein the one or more bioactive agents in the first phase promote a first tissue repair by selectively delivering the bioactive agent in the first implant phase to the first tissue and the one or more bioactive agents in the second implant phase promote a second tissue repair by selectively delivering the bioactive agent in the second implant phase to the second tissue.

35. The method of claim 34 wherein the first tissue is cartilage and the second tissue is bone.

36. The method of claim 35 wherein the one or more bioactive agents in the phase to promote cartilage repair comprises a suspension of chondrocytes.

37. The method of claim 30 further comprising inserting the two-phase implant into a delivery device.

38. The method of claim 37 further comprising inserting the implant into a defect in a patient.

39. The method of claim 38 wherein the defect in a patient spans bone tissue and cartilage tissue.
